(19) United States
(12) Patent Application Publication
(10) Pub. No.: US 2004/0059275 A1
(43) Pub. Date: Mar. 25, 2004

(54) Fat extraction

(76) Inventor: Kenneth Allan De Luca, Kyabram (AU)

Correspondence Address: LADAS & PARRY, 26 WEST 61ST STREET, NEW YORK, NY, 10023, US

(21) Appl. No.: 10/434,422

(22) Filed: May 7, 2003

Related U.S. Application Data
(63) Continuation of application No. PCT/AU01/01461, filed on Nov. 9, 2001.

(30) Foreign Application Priority Data
Nov. 9, 2000 (AU) PR1362
Jul. 2, 2001 (AU) PR6020
Sep. 7, 2001 (AU) PR7542

(51) Int. Cl. A61M 37/00; C02F 1/44; B01D 61/00

(52) U.S. Cl. 604/5.03; 604/4.01; 604/6.09; 604/6.11; 210/645; 210/668; 210/692

(57) ABSTRACT

The invention relates to a process of removing fat from a mammalian body comprising the steps of: extracting blood from said mammalian body, said blood containing solubilised fat; extracting at least a portion of said solubilised fat from the blood; and returning the extracted blood to the mammalian body. The invention also relates to an apparatus for performing the process and a method of treating obesity with the process and apparatus.

### FIELD OF THE INVENTION

The present invention relates to the removal of fat from the body of a mammal, in particular to a process and apparatus for removing fat from a mammalian body which involves passing blood outside the mammalian body and through a fat extraction means to remove blood borne fat. The invention further relates to a method of treating obesity in a mammal with the process and apparatus of the invention.

### BACKGROUND OF THE INVENTION

The mammalian body's means of storing and transporting fat is known as “lipid metabolism”. Fat ingested during a meal is absorbed and transported in the blood system to fat cells in the adipose tissue (called adipocytes), where it is absorbed and stored so that it can be used at a later time to provide energy to the body. Although fat cells may appear to be inert, they are in fact, metabolically very active and respond quickly to metabolic and hormonal stimuli, actively releasing and absorbing fat in a mechanism called “turn over”.

During “turn over”, the molecules of fat stored in a fat cell are released from storage and transported through the blood system and either used by an organ, such as the heart, to provide energy, or redeposited in adipose tissue elsewhere in the body. It has been estimated that as much as one half of the total stored fat in a body is turned over on a daily basis. The body has a natural tendency to achieve homeostasis and it will naturally try to distribute fat equally between all available fat cells.

Growing levels of obesity in our modern populations is a well known medical problem to which there have been many attempts to effect a suitable remedy. Treatments for obesity include stomach staples, calorie control diets, drugs and liposuction. However, most weight-loss programs have a failure rate of around 95% over two years, whether they involve diets, drugs or invasive medical procedures. There is a need for a non-invasive therapy that allows weight loss, particularly rapid weight loss, and reduces or eliminates the difficulties or side effects of current weight loss treatments.

### SUMMARY OF THE INVENTION

According to a first aspect of the present invention, there is provided a process for removing fat from a mammalian body comprising:

extracting blood from said mammalian body, said blood containing solubilised fat;

extracting at least a portion of said solubilised fat from the blood; and

returning the extracted blood to the mammalian body.

According to a second aspect of the present invention, there is provided an apparatus for removing solubilised fat from blood of a mammal comprising:

a pump to pump blood from the mammal; and

fat extraction means for separating solubilised fat from the blood pumped from the mammal to permit blood absent of the separated solubilised fat to be returned to the mammal.

According to a third aspect of the present invention, there is provided a method of treating obesity in a mammal comprising the steps of:

extracting blood from said mammal, said blood containing solubilised fat;

extracting at least a portion of said solubilised fat from the blood; and

returning the extracted blood to the mammal.

### DESCRIPTION OF THE PREFERRED EMBODIMENTS

The term “fat” as used herein includes but is not limited to fatty acids and esters of fatty acids and glycerol, such as mono-, di- and triglycerides. Triglycerides may be in the form of triacylglycerides and may be part of chylomicrons that transport triacylglycerols from the intestinal tract to the adipose tissue. The fatty acids that are released from adipose tissue are transported in the blood by serum albumin. Each serum albumin can transport two long chain fatty acids tightly bound to the protein and another one or two long chain fatty acids that are more loosely bound.

As used herein, the term “solubilised fat” refers to blood borne fat, that is, fatty acids and mono-, di- and triglycerides that are associated with carrier proteins so that they are soluble and can be transported in the blood. Carrier proteins include serum albumin, α-globulins and β-globulins. In particular, fatty acids are solubilised and transported in the blood by serum albumin.

The term “stored fat” as used herein refers to fatty acids which are stored as triglycerides in fat cells in a mammalian body. Fat is transported from the intestine to the fat cells where fatty acids are absorbed and stored as triglycerides. During “turn over” fatty acids are hydrolysed from the triglycerides by lipases in the fat cells and pass into the blood stream where they are bound by serum album and transported to an organ for use or reabsorbed for storage by another fat cell.

The term “fat cells” as used herein are also known as adipocytes and are the cells that make up the adipose tissue in a mammal. Fat cells actively store fat in the form of triglycerides and release fat in the form of fatty acids which are transported in the blood stream bound to serum albumin.

The term “adipose tissue” as used herein is a tissue or collection of adipocytes or fat cells which includes blood vessels such as arteries, veins and capillaries. The cells of the blood vessels have lipoprotein lipases that are able to hydrolyse fatty acids from triglycerides associated with chylomicrons so that the fatty acids may be absorbed by the fat cells of the adipose tissue.

The term “obesity” as used herein is defined as an excessive content of fat in adipose tissue. This term relates to mammals that have a body weight above what is considered to be a normal healthy weight and includes mammals that are generally overweight and those that are excessively overweight.

Throughout the specification and the claims which follow, unless the context requires otherwise, the word “comprise”, and variation such as “comprises” and “comprising”, will be understood to imply the inclusion of a stated integer or step or group of integers or steps but not the exclusion of any other integer or step or group of integers or steps.

In a broad form, the process of the invention relates to removing at least a portion of fat from a mammalian body by extracting solubilised fat from the blood of the mammalian body. In particular, the process enables the removal of stored fat from the mammalian body's fat stores in fat cells when the stored fat is released from the fat cells and transported around the body in the blood as solubilised fat, for use or redistribution among available fat cells. The fat is extracted by passing the blood through a fat extraction means and then returning the blood to the mammalian body.

The mammalian body may be a human body or an animal body. Preferably the mammalian body is a human body.

The blood may be extracted from the mammalian body by any suitable means. For example, blood may be extracted by inserting a cannula into a blood vessel of a mammalian body and allowing the blood to flow through tubing to the fat extraction means. Preferably, the blood is extracted using a pump to control the rate of flow of the blood being extracted. Preferably the pump is a blood pump and optionally more than one pump may be used. Similarly, the blood may be returned to the mammalian body by any suitable means. For example, the blood may be returned by way of tubing and cannula into another blood vessel after it has passed through the fat extraction means. The blood may be extracted from and returned to any suitable blood vessels. For example, the blood may be extracted from the radial artery and returned to the saphenous vein. Preferably, anticoagulants may be added to the blood extracted from the mammalian body.

The solubilised fat may be extracted from the blood by passing the blood through any suitable fat extraction means.

In one embodiment, the fat extraction means comprises a chamber containing a fat absorbing substance. Preferably, the fat absorbing substance comprises fat cells.

The fat cells may be donor fat cells derived from the mammalian body or another source, such as an animal, and grown in vitro. The fat cells may be generic fat cells that are compatible with the mammalian body being treated. Genetic fat cells are cells that are grown from suitable donor cells that are not from the mammalian body and may be compatible as they are suspended in blood of an appropriate blood type. Fat cells may be derived from donor fat cells obtained by biopsy of adipose tissue and cultured by well known tissue culture means, such as immersion in nutrient media and incubation. The cells prepared in this manner may be rinsed and isolated by centrifugation to form a cell pellet which may be resuspended in a suitable fluid, such as blood plasma, by gentle mechanical agitation before transfer to the fat extraction means of the invention. The fat cells may be cultured in a way to maintain the cells in individual form, or they may be cultured to produce tissue strips or clumps of associated cells.

Suitable fat cells may also be produced from adult or fetal stem cells which may be derived from a human or animal. Fat cells may also be harvested from an animal, such as a pig, in a similar manner to other organs that are harvested for transplant. The fat cells may also be genetically engineered human or animal fat cells that have been modified to increase their ability to absorb blood borne fat and/or to avoid immune response problems.

The fat cells may be individual fat cells, or they may be formed into membranes or tissue layers held in suspension in a chamber and through which the blood can pass. The membranes or tissues may include arteries, veins and capillaries, the growth of which have been stimulated by suitable hormones. The blood flow may be arranged to flow through the arteries, veins and capillaries to deliver the solubilised fat to the fat cells. Alternatively, the fat cells may be in the form of modified transplanted animal adipose tissue that has inherent blood vessels associated with it. The transplanted adipose tissue may be obtained, for example, from pigs that have been genetically developed to provide lean adipose tissue, or pigs that have been kept at or brought to an extremely lean state.

When the fat absorbing substance is adipose tissue, whether membranes or tissues having blood vessels or transplanted adipose tissue, the solubilised fat may be in the form of chylomicrons. The fat may be released from the chylomicron by lipoprotein lipases located in the cells of the blood vessels and absorbed into the fat cells of the tissue or membrane.

The fat absorbing substance, whether individual fat cells or in the form of membranes or adipose tissue, is suspended in the chamber. Preferably, the fat cells are suspended in blood from the mammalian body. The chamber has an inlet to allow the blood to flow in and an outlet downstream from the inlet, to allow the blood stream to flow out after it has passed through the fat absorbing substance. The chamber may be any suitable size, for example, it may be large enough to accommodate an amount of fat absorbing substance required to absorb fat for an entire treatment period. Alternatively, the chamber may be smaller and include a means of replacing the fat absorbing substance when it can no longer absorb solubilised fat during the treatment period.

In the case where individual fat cells are used as the fat absorbing substance, a means of keeping the cells in suspension may be required. Any suitable means of suspending the cells without damaging them or the blood cells may be used, for example, mechanical agitation. Such agitation can be achieved by well known means in the art, such as agitators that have rocking or circular motion, or agitators using a sterile magnetic stirrer bead which is motivated by an external magnet rotated by an electrical motor. Furthermore, the chamber may include a means of preventing the fat cells from leaving the chamber. Any suitable means of preventing the fat cells leaving the chamber in the blood may be used, for example, filters of appropriate pore size may be used. To ensure that the fat cells are not able to pass through the filter pores, they may be treated so that they have a minimum size. For example, the fat cells may be partially filled with fat or they may be induced during their preparation by tissue culture to associate with other fat cells in partial tissue formation. Many blood filtering technologies and materials are known in the art and could be applied in the present invention.

In a preferred embodiment, the chamber may contain individual fat cells in a flexible bladder, the flexible bladder allowing for the expansion of the volume of the fat cells as they absorb the solubilised fat from the blood. The flexible bladder may also be readily removed from the chamber allowing for the removal of full fat cells and their replacement with a new flexible bladder containing fresh empty fat cells. The flexible bladder may be made from any suitable flexible material that is biologically inert, for example, biologically inert elastic materials. Suitable materials include those used in blood storage bags, for example, polyvinylchloride (PVC). To maintain an optimal, effective quantity of blood to suspend the fat cells, control of the blood flowing in and out of the bladder may be provided. Control of blood flow may be achieved with suitable pressure and valve means, for example, the use of pumps such as blood pumps in combination with appropriate valves. The blood flow may be controlled by use of more than one pump, working together, for example, a pump which controls the flow of blood from the mammal and a pump located downstream from the fat extraction means which controls the flow of blood returning to the mammal.

In this embodiment, the process of the invention allows the blood to pass through a fat absorbing substance, preferably comprising fat cells, located externally from the mammalian body so that the solubilised fat may be absorbed by the fat absorbing substance. Given the body's natural tendency to achieve homeostasis, it will try to redistribute the solubilised fat equally between all available fat cells. Therefore if the number of fat cells available is increased by the addition of external fat cells, the stored fat in the body will be reduced as the fat is redistributed between the body fat cells and the external fat cells.

When the fat cells are full, they may be removed from the device and replaced with fresh empty fat cells and the process may be continued. This may allow the fat deposits in the mammalian body to halve in a very short time, such as hours or days.

In another embodiment, the fat extraction means comprises a filtration system including a first filter having a pore size to exclude blood components having a size greater than solubilised fat, a second filter having a pore size to exclude the solubilised fat and a means of recombining the blood components without the solubilised fat. The solubilised fat is preferably in the form of fatty acids bound to serum albumin. The blood is passed through the first filter and is separated into a large component flow and a first filtrate. The first filtrate is then passed through the second filter and a second filtrate and a solubilised fat flow are obtained. The second filtrate and the large component flow are then combined and returned to the body. The solubilised fat flow may be discarded or treated to remove the fat from the protein carrier.

Mixing or recombination of the blood components may occur by use of normal turbulence in the fat extraction means caused by pumping the blood around the means. However, turbulence inducing features within the fat extraction means may also be used. For example, the junction at which the large component flow and the second filtrate meet may be formed in such a way to provide turbulence suitable to result in mixing of the blood components.

The blood which has passed through the filtration system has a low fat and albumin concentration. To determine the concentration of blood components, the apparatus may include a means of sampling the recombined blood. Such means are known in the art and may include a tap or valve through which a syringe needle may be inserted. The blood samples taken in this manner may be tested to determine concentrations of blood components such as serum albumin or fat. To re-establish a normal concentration of serum albumin in the blood returning to the body, serum albumin which is free of fatty acids, may be added to the large particle flow, the second filtrate or their combination. The serum albumin, which is free of fatty acids, may be externally derived, or obtained by stripping the fatty acids from the serum albumin contained in the solubilised fat flow. Also a combination of externally derived fat free serum albumin and fat free serum albumin obtained by stripping the fat from the solubilised fat flow may be used. The apparatus may therefore include a means of adding additives, such as fat free serum albumin, to the large particle flow, second filtrate or their combination. Any suitable means known in the art may be used, for example, a controlled flow may be added by means of a three way tap or pump.

It is preferable to recycle the serum albumin removed from the blood with the solubilised fat. Therefore, the apparatus may include a means of stripping the fat from the serum albumin and separating the fat free serum albumin for return to the blood. The fat free serum albumin may be added directly to the second filtrate, the large particle flow or the combined blood, or may be isolated and added separately.

In yet another embodiment of the invention, the fat extraction means comprised a centrifuge. The blood is fractionated in the centrifuge according to density to provide a solubilised fat fraction, which is removed from the fractionated blood. The fractionated blood from which the solubilised fat fraction has been removed, is then mixed. The centrifuge may be a continuous centrifuge, similar in function to that of a cream separator, having internal settings such that a fraction of predetermined density, which contains the solubilised fat, may be continuously drawn off and separated from other blood components.

Alternatively, the blood may be separated into discrete quantities for centrifuging and the fraction containing the fat drawn off, the blood may be recombined or remixed before being returned to the body. This may be achieved by using a series of devices which fractionate, draw off the desired fraction and then remix the remaining components, such that the series provides a continuous flow.

This embodiment may be combined with a filtration step if the solubilised fat fraction includes other blood components that need to be returned to the mammalian body.

Again, in this embodiment of the invention, the resultant blood stream is depleted of serum albumin. Accordingly, the apparatus preferably includes a means of sampling the blood being returned to the mammal, a means of adding fat free serum albumin to the separated blood or remixed blood and/or a means of stripping the fat from the serum albumin so that the fat free serum albumin may be returned to the blood as described above.

It is known that the body may defend itself against toxins by binding them up in fat deposits. Therefore, it is preferred that the process of the invention is used in conjunction with a means for removing toxins from the blood, for example, a dialysis machine such as a kidney machine. This would remove toxins, waste products or impurities from the blood that may be released from the fat deposits in the mammalian body or otherwise resulting from the fat extraction process. The apparatus of the invention may include connection means so that a means for removing toxins may be attached. The connection means may be any suitable means of attachment known in the art. For example, the connection means may include an outlet, a inlet and a valve. The outlet may be, for example, a three way tap located upstream or downstream from the fat extraction means, that is able to be attached to the means for removing toxins and allows the blood to flow to the means for removing toxins or allows it to be bypassed. The inlet may be, for example, a three way tap downstream from the outlet, to allow the blood to flow from the means for removing toxins, back into the apparatus of the invention, or for the means for removing toxins to be bypassed. The valve, located between the inlet and the outlet, is closed when the blood is being directed to the means for removing toxins, and is open when the means for removing toxins is being bypassed. Preferably, connection means is located downstream from the fat extraction device.

In some forms of the present invention, such as the examples shown in FIGS. 1 and 2 and where optional toxin removal is utilised, the apparatus of the invention may be described in terms of a well known technology for hemodialysis, such as the hemodialysis kidney machine, which further includes a fat extraction means. The step of passing the patient's blood through a blood borne fat extraction means is then performed in conjunction with hemodialysis. Apparatus used in hemodialysis is well known in the art and may be utilized in conjunction with the present invention.

The apparatus of the invention may also include a means for removing bubbles of gas from blood, such as a bubble trap, located downstream from the fat extraction device to remove any gas bubbles in the blood before it is returned to the body.

The apparatus for removing solubilised fat from the blood of a mammal may be contained in a housing having an inlet to allow blood to be pumped into the apparatus and an outlet to allow blood to be returned to the body. The inlet and outlet may be attached to the mammal by any suitable means, for example, tubing and a cannula.

The process of the invention may also be used in conjunction with a therapy or treatment that enhances the release of fat stored in adipose tissue, effectively increasing the amount of “turn over” occurring in the body. This therapy or treatment may involve the manipulation of blood chemistry, such as manipulating the amount of serum albumin available to transport fatty acids released from adipose tissue, or insulin levels. The therapy or treatment may involve the administration of a compound or composition that enhances release of fat from adipose tissue into the blood stream, such as adrenaline.

To increase the transfer of stored fat in adipose tissue to the blood, a physical therapy may be applied. For example, the body being subjected to the process of the invention may be wrapped in tight pressure bandages or be treated with a device which applies an external pressure upon the patients fat cells. The external pressure provides a higher internal pressure within each fat cell relative to the body's blood pressure. By having a higher internal pressure inside the fat cell relative to the blood pressure outside the fat cell, the fat stored in the fat cell is more likely to pass across the membrane barrier to the blood and blood borne fat is less likely to pass back across the membrane barrier into the fat cells of the adipose tissue in the body. As this pressure differential does not exist in the fat extraction means described above, there would be a bias for more fat passing from the body's adipose tissue fat stores via the blood to the fat extraction means and a bias against the redeposit of fat in the body's fat cells and adipose tissue. The fat extraction means may enhance this bias by having reduced pressures inside the fat extraction means. Such a system may include applying a partial vacuum to the chamber containing the fat cells in the form of cultured or transplanted adipose tissue or to the filter system.

Advantageously, the process of the invention is applied together with careful blood flow planning that ensures that not all of the solubilised fat is removed from the blood or that some solubilised fat is replaced in the blood after the extraction process is complete. A level of solubilised fatty acid is required by the heart to provide energy therefore complete depletion of solubilised fat may place stress on the heart. Such blood flow planning may be important where the fat extraction means used in the process contains fat cells that are genetically engineered to aggressively take up fat from the blood. However, careful monitoring of the fat content of the blood stream returned to the body will enable management or elimination of this potential risk.

Given that the “turn over” of fat may involve half of the patient's stored fat daily, then the process of the invention may provide rapid and non invasive weight loss from the mammalian body.

The process of the invention may be used to treat obesity in humans or animals. While rapid weigh loss has historically been viewed as unhealthy, such weight loss was previously effected by placing huge stress on the body, for example, large reductions in blood glucose levels. The process of the present invention can be used to effect rapid weight loss without placing undue stress on the body. The body redistributes its fat stores on a daily basis and in the process of the invention redistributes fat to the fat extraction means which is externally located, normal metabolism is therefore maintained.

Although the rapid weight loss may be desirable for cosmetic reasons, there are many health risks associated with obesity, for example, increased risk of heart disease, stroke, hypertension and diabetes. Weight loss effected by the treatment of the present invention will reduce these risks. The treatment of the present invention is also useful for rapid weight loss in obese patients that need surgery, reducing the risks associated with surgery and the difficulties associated with the presence of gross fat deposits. The treatment of the invention is also useful for obese people who have suffered strokes or are incapacitated, and they are too heavy for normal rehabilitation thereby hindering their ability to regain mobility. The treatment of the present invention may also be useful in conjunction with a treatment for overeating, such as counselling and nutritional education. If an obese person rapidly loses weight, they may be able to participate in exercise programs and feel good about themselves promoting beneficial mental health and maintenance of their reduced body weight.

The person to be treated with the invention may donate blood about a week before the treatment to provide enough blood to make up the requisite quantities for the tubing and for suspension of the fat cells in the chamber.

### BRIEF DESCRIPTION OF THE DRAWINGS

FIG. 1 is a schematic representation of an apparatus and process of the invention where the fat extraction means includes a chamber containing fat cells.

FIG. 2 is a schematic representation of an apparatus and process of the invention where the fat extraction means includes a filtration means.

### DETAILED DESCRIPTION

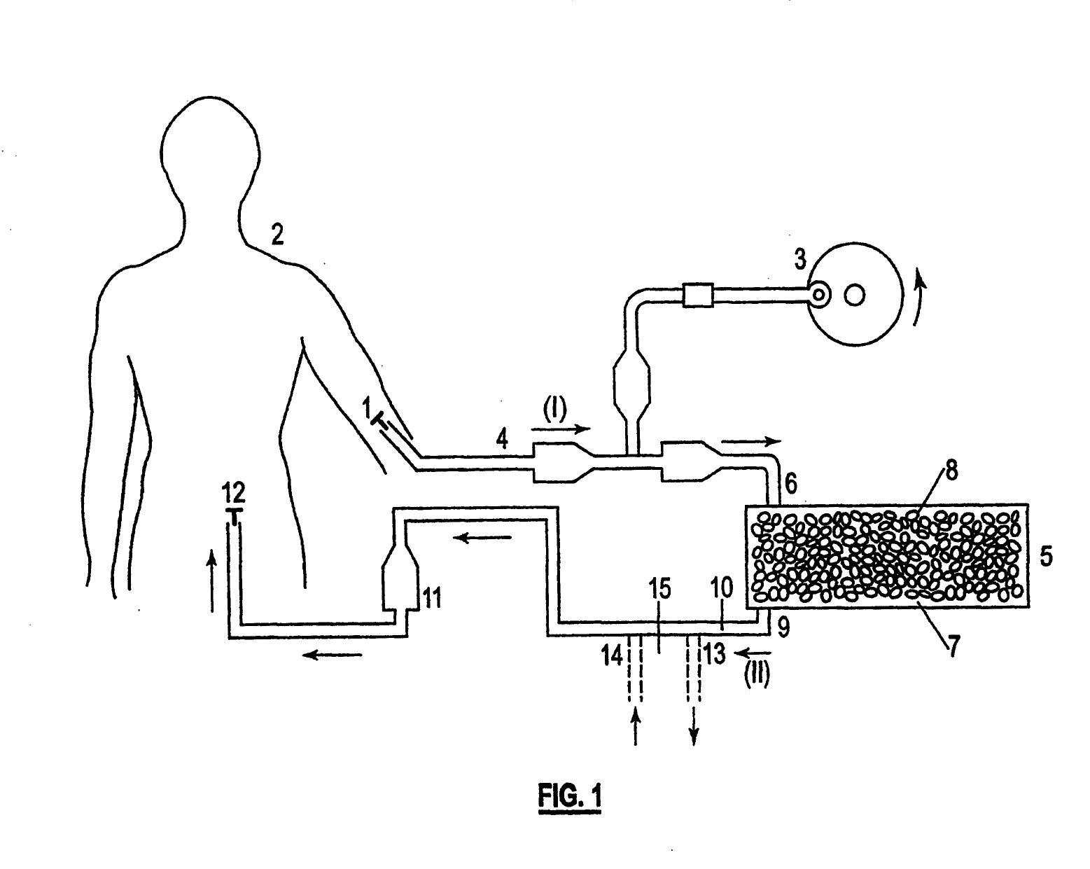

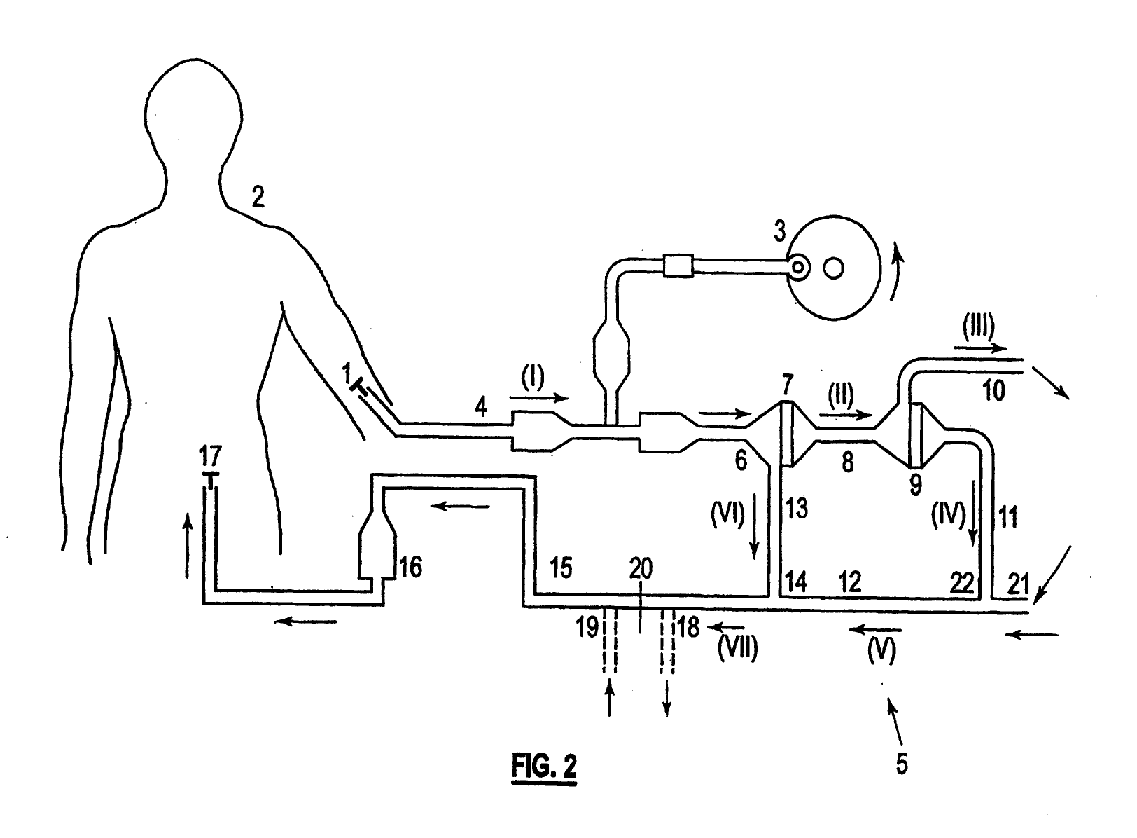

Preferred embodiments of the apparatus and process of the invention will now be described with reference to FIGS. 1 and 2.

Referring to FIG. 1, a cannula (1) is inserted into the radial artery of a human body (2) to allow blood to be pumped by blood pump (3) in the direction of arrow (I) through a first tubing (4). The blood stream is pumped into the fat extraction means (5) through an inlet (6). The fat extraction means comprises a chamber (7) which contains a suspension of fat cells (8). The blood is allowed to flow through the fat cells (8) and exit the chamber through outlet (9) and into a second tubing (10) in the direction of arrow (II). Before returning to the body (2), the blood is passed through a bubble trap (11) to remove any air bubbles. The blood is then allowed to re-enter the body by way of a cannula (12) inserted into the saphenous vein of the body (2). The apparatus also shows an optional point of attachment for a means for removing toxins. Tubing (10) has an outlet (13) (shown in the closed position) which if open would allow blood to flow from tubing (10) to the means for removing toxins. Downstream from the outlet (13) is an inlet (14) (shown in the closed position) which if open would allow blood to flow from the means for removing toxins back into tubing (10). Between the outlet (13) and the inlet (14) is a valve (15). When the outlet (13) and the inlet (14) are closed and the valve (15) is open, the blood bypasses the means for removing toxins. When the outlet (13) and the inlet (14) are open and the valve (15) is closed, the blood flows through the means for removing toxins.

Referring to FIG. 2, a cannula (1) is inserted into the radial artery of a human body (2) to allow blood to be pumped by blood pump (3) in the direction of arrow (I) through a first tubing (4). The blood is pumped into the fat extraction means (5) through an inlet (6). The fat extraction means comprises a first filter (7) having a pore size suitable to exclude particles larger than solubilised fat. The blood is separated at the first filter (7) into a large component flow and a first filtrate. The first filtrate passes into tubing (8) and flows in the direction of arrow (II) to a second filter (9). The second filter (9) has a pore size suitable to exclude particles the size of solubilised fat. The first filtrate is separated at the second filter (9) into a solubilised fat flow and a second filtrate. The solubilised fat flow is syphoned away from the second filter (9) through tubing (10) in the direction of arrow (III) and is either pumped to waste or to a treatment system to remove and separate the fat from the carrier protein. The second filtrate flows into tubing (11) in the direction of arrow (IV) and flows through tubing (12) in the direction of arrow (V) where it is combined with the large component flow. The large component flow is syphoned away from the first filter (7) into tubing (13) in the direction of arrow (VI) where it combines with the second filtrate at tubing junction (14). The combined second filtrate and large component flow form a blood stream that flows through tubing (15) in the direction of arrow (VII). Before returning to the body (2), the blood stream is passed through a bubble trap (16) to remove any air bubbles. The blood stream is then allowed to re-enter the body by way of a cannula (17) inserted into the saphenous vein of the body (2). The apparatus also shows an optional point of attachment for a means for removing toxins. Tubing (15) has an outlet (18) (shown in the closed position) which if open would allow blood to flow from tubing (15) to the means for removing toxins. Downstream from the outlet (18) is an inlet (19) (shown in the closed position) which if open would allow blood to flow from the means for removing toxins back into tubing (15). Between the outlet (18) and the inlet (19) is a valve (20). When the outlet (18) and the inlet (19) are closed and the valve (20) is open, the blood bypasses the means for removing toxins. When the outlet (18) and the inlet (19) are open and the valve (20) is closed, the blood flows through the means for removing toxins. The fat extraction device (5) also includes an inlet (21) in which fat free serum albumin may be combined with the second filtrate at tubing junction (22). The fat free serum albumin may be obtained from external sources, or may be the product of the treatment of the protein-bound fat flow.

The claims defining the invention are as follows:

1. A process of removing fat from a mammalian body comprising the steps of:
extracting blood from said mammalian body, said blood containing solubilised fat;
extracting at least a portion of said solubilised fat from the blood by exposing the blood to fat cells; and
returning the extracted blood to the mammalian body.

2. A process according to claim 1 wherein the fat cells are contained in a chamber.

3. A process according to claim 1 wherein the fat cells are derived from donor fat cells from the mammalian body.

4. A process according to claim 1 wherein the fat cells are generic fat cells that are compatible with the mammalian body.

5. A process according to claim 1 wherein the fat cells are produced from adult or fetal, animal or human stem cells.

6. A process according to claim 1 wherein the fat cells are genetically engineered human or animal fat cells modified to increase their fat absorbing properties and/or immune response properties.

7. A process according to claim 1 wherein the fat cells are in the form of individual fat cells.

8. A process according to claim 1 wherein the fat cells are in the form of membranes or cultured tissue layers.

9. A process according to claim 8 wherein the membrane or tissue comprises hormone stimulated arteries, capillaries and veins.

10. A process according to claim 1 wherein the fat cells are in the form of modified animal adipose tissue which contains blood vessels.

11. A process according to claim 7 further comprising the step of mechanically agitating the fat cells within the chamber.

12. A process according to claim 2 wherein the step of extracting at least a portion of the solubilised fat further comprises:
passing the blood containing solubilised fat through a first filter for separating any blood components larger than the fat cells from the blood thereby forming a large component flow and a first filtrate,
passing said first filtrate through said chamber or a portion of said chamber containing fat cells to separate solubilised fat from said first filtrate thereby forming a second filtrate,
passing said second filtrate through a second filter capable of retaining fat cells thereby forming a third filtrate, and
combining said large component flow with said third filtrate.

13. A process according to claim 1 wherein the mammalian body is a human body.

14. A process according to claim 1 comprising the further step of passing the blood through a means for removing toxins from the blood.

15. A process according to claim 1 comprising the further step of treating the mammalian body to enhance the release of fat from adipose tissue into the blood stream.

16. A process according to claim 15 wherein the treatment comprises administration of a compound or composition that enhances release of stored fat from adipose tissue into the blood.

17. A process according to claim 15 wherein the treatment comprises applying external pressure to the mammalian body to provide a higher internal pressure in the fat cell relative to the body's blood pressure.

18. An apparatus for removing fat from blood of a mammal comprising:
a pump to pump blood from the mammal; and
fat extraction means for separating solubilised fat from the blood of the mammal to permit blood absent the separated fat to be returned to the mammal, said fat extraction means comprising fat cells.

19. An apparatus according to claim 18 wherein the fat cells are contained in a chamber through which solubilised fat from the blood can flow, but from which the fat cells cannot escape.

20. An apparatus according to claim 18 further comprising a means of mechanically agitating the individual fat cells within the chamber.

21. An apparatus according to claim 19 wherein the chamber further comprises a flexible bladder in which the fat cells are located.

22. An apparatus according to claim 21 wherein said apparatus further comprises a second blood pump located downstream from the fat extraction device.

23. An apparatus according to claim 19 wherein the chamber further comprises a filter to prevent the fat cells leaving the chamber.

24. An apparatus according to claim 19 wherein the chamber further comprises a means of removing and/or replacing the fat cells.

25. An apparatus according to claim 18 wherein the fat extraction device further comprises means for adding additional fat free serum albumin to the blood passing back to the mammal which is absent the separated solubilised fat.

26. An apparatus according to claim 18 further comprising means for sampling blood before it is returned to the body.

27. An apparatus according to claim 18 further comprising means for removing toxins from the blood.

28. A method of treating obesity in a human comprising the steps of:
extracting blood from said human, said blood containing solubilised fat;
extracting at least a portion of said solubilised fat from said blood by exposing said blood to fat cells; and
returning the extracted blood to said human.

29. A method of treating obesity in a human comprising the steps of:
extracting blood from said human, said blood containing solubilised and/or insolubilised fat;
extracting at least a portion of said solubilised and/or insolubilised fat from said blood by exposing said blood to a means for removing said solubilised and/or insolubilised fat; and
returning the extracted blood to said human.
